(19) United States
(12) Patent Application Publication
(10) Pub. No.: US 2026/0232718 A1
(43) Pub. Date: Aug. 13, 2026

(54) INJECTABLE FORMULATION

(71) Applicant: Meta Healthcare Ltd, Gloucestershire (GB)

(72) Inventors: Stephen Tickle, Frodsham (GB); Christopher Wagstaff, Cambridge (GB)

(73) Assignee: Meta Healthcare Ltd, Gloucestershire (GB)

(21) Appl. No.: 19/285,911

(22) Filed: Jul. 30, 2025

(30) Foreign Application Priority Data
Feb. 10, 2025 (GB) 2501964.7

(51) Int. Cl. A61K 31/714; A61K 9/00; A61K 9/08; A61K 47/02; A61K 47/12

(52) U.S. Cl. CPC A61K 31/714; A61K 9/0019; A61K 9/08; A61K 47/02; A61K 47/12

(57) ABSTRACT

This disclosure relates to an injectable formulation and the use of the formulation in a method of treatment. The injectable formulation is intended for use in the treatment of children and adults with genetic defects of intracellular cobalamin (cbl) processing, particularly the cblC disorder.

### CROSS-REFERENCE TO RELATED APPLICATION

This application claims priority to Great Britian Patent Application No. 2501964.7 filed 10 Feb. 2025, entitled INJECTABLE FORMULATION, the disclosure of which is hereby incorporated herein by reference.

### TECHNICAL FIELD

This disclosure relates to an injectable formulation and the use of the formulation in a method of treatment. The injectable formulation is intended for use in the treatment of children and adults with genetic defects of intracellular cobalamin (cbl) processing, particularly the cblC disorder.

### BACKGROUND

There are currently 8 known ultra rare genetic disorders of intracellular cobalamin processing; these have been classified as groups cblA, B, C, D, E, F, G, and J by complementation phenotyping of fibroblasts. More recently, three additional distinct genetic defects affecting transcriptional regulation have been discovered that mimic the cblC disorder: the X-linked cblX disorder and further two genetic defects that have been named cblK disorder. All these disorders are caused by recessive pathogenic variants of at least 12 different genes that encode or regulate the transcription of proteins involved in intracellular transport and activation of cobalamin and are characterised by reduced intracellular availability of either or both of the two active forms of cobalamin, adenosylcobalamin and methylcobalamin, which are required as co-factors for the enzymes methylmalonyl-CoA mutase and methionine synthase, respectively. A deficiency in one or more of adenosylcobalamin and methylcobalamin is referred to herein as “functional cobalamin deficiency.”

Genetic disorders of intracellular cobalamin present a broad spectrum of severity; the majority of cases however manifest during the neonatal period or in early infancy. Neonates present with a life-threatening multisystem disorder including megaloblastic anaemia, thrombotic microangiopathy and encephalopathy. During early infancy the disorders can manifest with failure to thrive, megaloblastic anaemia, atypical haemolytic uraemic syndrome and pulmonary hypertension, developmental delay, intellectual deficit, seizures and rapidly progressing retinopathy. Eye disease is a prominent clinical symptom. A large case series of children with the cblC disorder reported prevalences of nystagmus (64%), strabismus (52%), macular degeneration (72%), optic nerve pallor (68%), and vascular changes (64%). Children with the most common cblC genotype, MMACHC c.271dupA (p.R91KfsX14) homozygous, show early and extensive macular degeneration.

Children older than 1 year, adolescents and adults with milder, late-onset variants are increasingly being diagnosed. They predominantly present with variable neurological symptoms, including developmental regression, movement disorders, spastic paraplegia, seizures, dementia, or psychosis but can also present with thrombotic microangiopathy.

Disorders of intracellular cobalamin processing are usually uncovered by finding increased levels of methylmalonic acid (MMA) and/or homocysteine (Hcy) in body fluids, in the presence of normal plasma levels of cobalamin and folate. The diagnosis is confirmed by genetic testing. According to biochemical characteristics, they can be grouped in three categories: an isolated increase in MMA is found in cblA cblB and cblD-MMA disorder, an isolated increase in Hcy is seen in cblD-HC, cblE, and cblG, whereas both MMA and Hcy are increased in the so-called combined defects cblC, cblD-MMA/HC, cblF, and cblJ. The cblC disorder is the most common of all the disorders.

Cobalamin is derived from microbial metabolism and is available to humans from animal food sources. Dietary requirements of cobalamin for healthy adults and infants are 1.4 μg and 0.4 μg per day, respectively. Intestinal uptake and bioavailability rely on a high-affinity-low-capacity receptor-mediated specific transport system. Additional cobalamin can be absorbed through passive diffusion with an estimated uptake of 1-2% of an orally administered dose.

Nutritional cobalamin deficiency can be treated with oral supplements or a single parenteral administration to replete hepatic cobalamin stores, whereas disorders of cobalamin absorption or extracellular transport are associated with a decreased capacity to conserve cobalamin and usually require parenteral administration at increased frequency. It is estimated that approximately 10-15% of injected cobalamin is retained in the organism.

Two different forms of administration for intramuscular cobalamin are available: cyanocobalamin and hydroxocobalamin. Hydroxocobalamin is suggested by the British Pharmacopoeia as preferred option for intramuscular administration.

Parenteral hydroxocobalamin has very few reported adverse effects at high doses. Red discoloration of the urine and skin are expected at high doses. Hypersensitivity and photosensitivity reactions, nausea, infusion site reactions and headache are less common. The safety of doses up to 10,000 mg was assessed in a randomised placebo-controlled study in adults. Acutely, 5,000 to 10,000 mg of intravenous hydroxocobalamin are given as short infusion for the treatment of acute cyanide poisoning.

International guidance, following longstanding clinical practice, suggests treating newborn babies presenting with severe manifestations of disorders of intracellular cobalamin processing with daily parenteral doses of hydroxocobalamin, typically 1 mg intramuscular (IM) or intravenous (IV), corresponding to 0.3 mg/kg body weight during the first weeks of life. After stabilization, the frequency of administration is often decreased to minimize the distress from IM injections and the dose is frequently not increased to account for weight gain. This practice provides progressively lower cobalamin serum levels that may become sub-therapeutic.

There is increasing evidence from case studies that high-dose and high-frequency parenteral treatment is beneficial for clinical outcomes, as expected from the pharmacokinetic profile of parenteral hydroxocobalamin. Marked clinical and neurological deterioration has been described in patients with the cblC disorder that were weaned from daily to less frequent dosing. Increasing the parenteral dose of hydroxocobalamin to 5 mg daily helped in the resolution of thrombotic microangiopathy in two 13- and 8-year-old patients with the cblC disorder.

Genetic disorders of intracellular cobalamin processing typically present with life-threatening or debilitating complications. Although treatment with supraphysiological doses of parenteral cobalamin is generally effective to resolve acute haematological and vascular manifestations, other manifestations are only partially responding to the current standard of care. Cognitive impairment and progression of retinopathy is commonly observed on current cobalamin dosing regimens and there remains a risk of relapse of thrombotic microangiopathy. In a single centre evaluation, all 11 children with cblC disorder diagnosed after clinical manifestation and 9 out of 15 children diagnosed by newborn screening showed global developmental delay or cognitive dysfunction as well as a high prevalence of seizures, stroke, retinopathy, anemia, and cerebral atrophy, despite standard treatment.

### SUMMARY

Therefore, in certain embodiments of the present disclosure, there is an aim to provide a formulation for improving cognitive impairment and reducing the progression of retinopathy. In addition, certain embodiments of the present disclosure aim to avoid thrombotic microangiopathy.

The licensed preparations of parenteral hydroxocobalamin available on the European market have concentrations between 1 mg/ml and 5 mg/ml and are not approved in the proposed indication. These concentrations allow a maximum dose of up to 5 mg for single IM injections. Frequent IM injections are poorly accepted by patients and parents because of the amount of pain caused by the injection. Subcutaneous doses can be administered more frequently, especially when using a subcutaneous catheter for bolus injections, or slow infusions but volumes for subcutaneous bolus administration are even more restricted, especially in young children.

The present disclosure provides an ultra-high-dose parenteral treatment allowing daily administration of 1-2 mg/kg bodyweight, it is an aim of the present disclosure to provide improved outcomes for patients compared to currently licensed treatments.

Patients administered hydroxocabalamin injections struggle to adhere to their medication regime. Injections are required daily and under current licensed treatments, the several injections per day can be required. The injections are incredibly painful and high volume of injection prolong the time of the painful injection. Therefore, the present disclosure aims to increase the adherence of patients to therapeutic regimens by providing an ultra high dose. As such, an aim of certain embodiments of the present disclosure is to increase compliance with treatment regimens and/or to increase compliance with daily injections of a therapeutic dose of hydroxocobalamin.

In accordance with the present disclosure there is provided an injectable hydroxocobalamin formulation comprising from 20 mg/ml to 100 mg/ml of hydroxocobalamin or a pharmaceutically acceptable salt thereof.

In a further aspect of the present disclosure, there is provided an injectable hydroxocobalamin formulation comprising from 20 mg/ml to 100 mg/ml of hydroxocobalamin or a pharmaceutically acceptable salt thereof for use in the treatment of cobalamin deficiency in genetic defects of intracellular cobalamin processing.

In a further aspect there is provided a method of treating cobalamin deficiency in genetic defects of intracellular cobalamin processing by administering an injectable hydroxocobalamin formulation comprising from 20 mg/ml to 100 mg/ml of hydroxocobalamin or a pharmaceutically acceptable salt thereof.

In certain embodiments, the injectable hydroxocobalamin formulation comprises from 20 mg/ml to 60 mg/ml of hydroxocobalamin of a pharmaceutically acceptable salt thereof. In certain embodiments, the injectable hydroxocobalamin formulation comprises from 25 mg/ml to 50 mg/ml of hydroxocobalamin of a pharmaceutically acceptable salt thereof. In certain embodiments, the injectable hydroxocobalamin formulation comprises 25 mg/ml of hydroxocobalamin of a pharmaceutically acceptable salt thereof. In certain embodiments, the injectable hydroxocobalamin formulation comprises 50 mg/ml of hydroxocobalamin of a pharmaceutically acceptable salt thereof. In certain embodiments, the injectable hydroxocobalamin formulation comprises 100 mg/ml of hydroxocobalamin of a pharmaceutically acceptable salt thereof.

In certain embodiments, the injectable hydroxocobalamin formulation is a liquid. The injectable hydroxocobalamin formulation may further comprise a variety of components; however, the balance of the injectable formulation will be water.

In certain embodiments, the hydroxocobalamin is hydroxocobalamin acetate.

The injectable hydroxocobalamin formulation may further comprise a pH modifier. The pH modifier may be selected from any pharmaceutically acceptable acid. The pH modifier may be acetic acid, for example glacial acetic acid.

The injectable hydroxocobalamin formulation may further comprise a second pH modifier. The second pH modifier may be selected from any pharmaceutically acceptable base. The second pH modifier may be sodium hydroxide.

The injectable hydroxocobalamin formulation may further comprise sodium chloride.

In an embodiment, the injectable hydroxocobalamin formulation comprises a pH modifier, a second pH modifier, and sodium chloride, with the balance of the injectable formulation being water.

In certain embodiments, the treatment of functional cobalamin deficiency in genetic defects of intracellular cobalamin processing is the treatment of homocystinuria with or without methylmalonic acidemia due to genetic defects of intracellular cobalamin processing or the treatment of homocystinuria with or without methylmalonic acidemia due to cobalamin C disorder.

In certain embodiments, the treatment of functional cobalamin deficiency in genetic defects of intracellular cobalamin processing is the treatment of: methylmalonic acidemia type cblA; methylmalonic acidemia type cblB; methylmalonic acidemia with homocystinuria type cblC; methylmalonic acidemia with homocystinuria type cblD; methylmalonic acidemia with homocystinuria type cblF; methylmalonic acidemia with homocystinuria type cblJ; methylmalonic acidemia with homocystinuria type cblX; homocystinuria type cblD-HC; homocystinuria type cblE; or homocystinuria type cblG.

In certain embodiments, the injectable hydroxocobalamin formulation is for parenteral use, for example intramuscular or subcutaneous injection.

### DETAILED DESCRIPTION

International guidance, developed from longstanding clinical practice, suggests treating newborn babies presenting with severe manifestations of disorders of intracellular cobalamin processing with daily parenteral doses of hydroxocobalamin, typically 1 mg IM or IV, corresponding to 0.3 mg/kg body weight during the first weeks of life. Treatment with supraphysiological doses of parenteral cobalamin is generally effective to resolve acute haematological and vascular manifestations linked with the condition, other manifestations are only partially responding to the current standard of care. Cognitive impairment and progression of retinopathy [Fuchs et al. (2012), “Ocular manifestations of cobalamin C type methylmalonic aciduria with homocystinuria.” Journal of American Association for Pediatric Ophthalmology and Strabismus 16, 370-375; Gizicki et al. (2014) “Long-term Visual Outcome of Methylmalonic Aciduria and Homocystinuria, Cobalamin C Type.” Ophthalmology 121, 381-386, Weisfeld-Adams et al. (2015) “Ocular disease in the cobalamin C defect: A review of the literature and a suggested framework for clinical surveillance.” Molecular Genetics and Metabolism 114, 537-546, Ku et al. (2016) “Spectrum of ocular manifestations in cobalamin C and cobalamin A types of methylmalonic acidemia.” Ophthalmic Genetics 37, 404-414] is commonly observed on current cobalamin dosing regimens and there remains a risk of relapse of thrombotic microangiopathy [Lemoine et al. (2018) “Cobalamin C Deficiency Induces a Typical Histopathological Pattern of Renal Arteriolar and Glomerular Thrombotic Microangiopathy.” Kidney International Reports 3, 1153-1162]. In a single centre evaluation, all 11 children with cblC disorder diagnosed after clinical manifestation and 9 out of 15 children diagnosed by newborn screening showed global developmental delay or cognitive dysfunction as well as a high prevalence of seizures, stroke, retinopathy, anemia, and cerebral atrophy, despite standard treatment [Bourque et al. (2021) “Outcomes of patients with cobalamin C deficiency: A single center experience.” JIMD Reports 57, 102-114]. In certain embodiments, the present disclosure aims to improve outcomes for the manifestations of these mentioned conditions.

### Example 1—25 mg/ml

### Formulation and Presentation

The formulation is a solution containing Hydroxocobalamin as (acetate), sodium chloride, acetic acid or sodium hydroxide for pH adjustment and water. The quantitative formulation is detailed below:
Hydroxocobalamin 25 mg/ml Formulation

| | Concentration |
|---|---|
| Raw Materials | % w/v |
| Hydroxocobalamin Acetate | 2.5 (as Hydroxocobalamin) |
| Sodium Chloride (Ph. Eur) | 0.8 |
| Sodium Hydroxide (Ph. Eur) | For pH Adjustment |
| Glacial Acetic Acid (Ph. Eur) | For pH Adjustment |
| Water for Injection Ph. Eur | To 100 |

The formulation can be made in the same way as Example 2.

The container closure system is comprised of a type I 1 ml colourless glass ampoule.

Secondary Packaging may consist of the following: Ampoule Label; and Carton (5 ampoules per box) or 1 ml syringe for example with 0.1 ml graduation.

### In-Process Specification

| Requirement | Specification |
|---|---|
| Appearance | Red Clear Solution |
| pH | 4.3-4.7 |

### Finished Product Specification

| Requirement | | Specification |
|---|---|---|
| Appearance | | Red Clear Solution |
| Identification | A) | Retention time of sample is concordant with |
| | | that of the reference standard. |
| | B) | To be Determined |

| | |
|---|---|
| Hydroxocobalamin | 95-105% label claim |
| Assay | |
| Related Substances | To be defined |
| PH | 4.3-4.7 |
| Particulate Matter | Confirms to current BP requirements |
| Sterility | Conforms to current BP requirements |
| Bacterial Endotoxins | Not greater than 6 EU/ml |

### Stability Studies

### Analytical Testing Schedule

| | Timepoints | | | | | | | |
|---|---|---|---|---|---|---|---|---|
| Storage Conditions | Initial | 1 | 3 | 6 | 9 | 12 | 18 | 24 |
| 25° C. ± 2° C./60% RH ± 5% RH | A, B | A | A | A | A | A | A | A, B |
| 40° C. ± 2° C./75% RH ± 5% RH | | A | A | A | | | | |

See table below for explanation of type A and B tests.

| Test Description | Specification | Method Ref |
|---|---|---|
| Appearance | Clear Red Solution | Visual |
| pH | 4.3-4.7 | |
| Hydroxocobalamin Assay | 95-105% Label Claim | |
| Related Substances | To be Defined | |
| Sub Visible Particles | Complies to BP | BP |
| Bacterial Endotoxins | Not Greater than 6 EU/ml | BP |
| Sterility | Complies to BP | BP |

| Test Description | Method Ref | A | B |
|---|---|---|---|
| Appearance | Visual | ✓ | |
| pH | | ✓ | |
| Hydroxocobalamin | | ✓ | |
| Assay | | | |
| Related Substances | | ✓ | |
| Sub Visible Particles | BP | ✓ | |
| Bacterial Endotoxins | BP | | ✓ |
| Sterility | BP | | ✓ |

### Example 2—50 mg/ml

| | | |
|---|---|---|
| Strength | 50 | mg/ml |
| Batch Size (ml) | 100 | |

| Raw materials | % w/v | Amount required/g |
|---|---|---|
| Purified water Ph. Eur | 93.4775 | 93.4775 |
| Hydroxocobalamin Acetate Ph. Eur | 5.7225 | 5.7225 |
| Sodium Chloride Ph. Eur | 0.8 | 0.8 |
| | 100 | 100 |

pH 4.3-4.7 with acetic acid

### Manufacturing Process

1—Transfer approx 75 ml of water to the vessel
2—Add sodium chloride and stir to dissolve
3—Add the hydroxocobalamin and stir to dissolve
4—Add 1M acetic acid to adjust pH
5—Measure the pH and adjust pH to 4.3 to 4.7
6—Dilute to volume with water and mix
7—Remeasure the pH
8—Fill into 100 ml amber glass bottle and seal with tamper-evident/child resistant (TE/CR) closure

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

What is claimed is:

1. An injectable hydroxocobalamin formulation comprising from 20 mg/ml to 100 mg/ml of hydroxocobalamin or a pharmaceutically acceptable salt thereof.

2. The injectable hydroxocobalamin formulation of claim 1, wherein the injectable hydroxocobalamin formulation comprises from 20 mg/ml to 60 mg/ml of hydroxocobalamin or a pharmaceutically acceptable salt thereof.

3. The injectable hydroxocobalamin formulation of claim 1, wherein the injectable hydroxocobalamin formulation comprises from 25 mg/ml to 50 mg/ml of hydroxocobalamin or a pharmaceutically acceptable salt thereof.

4. The injectable hydroxocobalamin formulation of claim 1, wherein the injectable hydroxocobalamin formulation comprises about 25 mg/ml or about 50 mg/ml of hydroxocobalamin or a pharmaceutically acceptable salt thereof.

5. The injectable hydroxocobalamin formulation of claim 1, wherein the injectable hydroxocobalamin formulation is a liquid.

6. The injectable hydroxocobalamin formulation of claim 1, wherein the hydroxocobalamin is hydroxocobalamin acetate.

7. The injectable hydroxocobalamin formulation of claim 1, further comprising a pH modifier.

8. The injectable hydroxocobalamin formulation of claim 1, further comprising a pharmaceutically acceptable acid.

9. The injectable hydroxocobalamin formulation of claim 1, further comprising sodium chloride.

10. A method of treatment, wherein the method is for treatment of functional cobalamin deficiency in genetic defects of intracellular cobalamin processing, wherein the method comprises administering a therapeutic amount of an injectable hydroxocobalamin formulation comprising from 20 mg/ml to 100 mg/ml of hydroxocobalamin or a pharmaceutically acceptable salt thereof.

11. The method of claim 10, wherein the treatment of functional cobalamin deficiency in genetic defects of intracellular cobalamin processing is the treatment of homocystinuria due to genetic defects of intracellular cobalamin processing.

12. The method of claim 10, wherein the treatment of functional cobalamin deficiency in genetic defects of intracellular cobalamin processing is the treatment of: methylmalonic acidemia type cblA; methylmalonic acidemia type cblB; methylmalonic acidemia with homocystinuria type cblC; methylmalonic acidemia with homocystinuria type cblD; methylmalonic acidemia with homocystinuria type cblF; methylmalonic acidemia with homocystinuria type cblJ; methylmalonic acidemia with homocystinuria type cblX; homocystinuria type cblD-HC; homocystinuria type cblE; or homocystinuria type cblG.

13. The method of claim 10, wherein the injectable hydroxocobalamin formulation comprises from 20 mg/ml to 60 mg/ml of hydroxocobalamin or a pharmaceutically acceptable salt thereof.

14. The method of claim 10, wherein the injectable hydroxocobalamin formulation comprises from 25 mg/ml to 50 mg/ml of hydroxocobalamin or a pharmaceutically acceptable salt thereof.

15. The method of claim 10, wherein the injectable hydroxocobalamin formulation comprises about 25 mg/ml or about 50 mg/ml of hydroxocobalamin or a pharmaceutically acceptable salt thereof.

16. The method of claim 10, wherein the injectable hydroxocobalamin formulation is a liquid.

17. The method of claim 10, wherein the pharmaceutically acceptable salt of hydroxocobalamin is hydroxocobalamin acetate.

18. The method of claim 10, wherein the injectable hydroxocobalamin formulation further comprises a pH modifier.

19. The method of claim 10, wherein the injectable hydroxocobalamin formulation further comprises a pharmaceutically acceptable acid.

20. A method of treating a patient with homocystinuria and methylmalonic acidemia due to the cblC phenotype disorder of intracellular cobalamin processing, the method comprising administering to the patient an injectable hydroxocobalamin formulation comprising from 20 mg/ml to 100 mg/ml of hydroxocobalamin or a pharmaceutically acceptable salt thereof.
